(19) United States
(12) Patent Application Publication
(10) Pub. No.: US 2017/0066701 A1
(43) Pub. Date: Mar. 9, 2017

(54) METAL ALKOXIDE AND A PROCESS FOR ITS PREPARATION

(71) Applicant: RELIANCE INDUSTRIES LIMITED, Mumbai (IN)

(72) Inventors: Virendrakumar GUPTA, Navi Mumbai (IN); Yogeshwar Narayanrao THAKARE, Amravati (IN); Bhavesh Kiritbhai DESAI, Surat (IN); Suketu VAKIL, Mumbai (IN)

(73) Assignee: RELIANCE INDUSTRIES LIMITED, Mumbai (IN)

(21) Appl. No.: 15/355,457

(22) Filed: Nov. 18, 2016

Related U.S. Application Data
(63) Continuation of application No. PCT/IB2015/053699, filed on May 20, 2015.

(30) Foreign Application Priority Data
May 20, 2014 (IN) 1694/MUM/2014

(51) Int. Cl. C07C 29/70; C07C 31/30

(52) U.S. Cl. CPC C07C 29/70; C07C 31/30

(57) ABSTRACT

The present disclosure relates to microporous magnesium alkoxide and its preparation. The magnesium alkoxide of the present disclosure is characterized by mean particle size ranging from 20 to 70μ; surface area ranging from 1 to 30 m²/g; circularity ranging from 0.5 to 0.9; macro pore size distribution ranging from 40 to 80%; meso pore size distribution ranging from 15 to 60%; and micro pore size distribution ranging from 2 to 10%.

### CROSS REFERENCE TO RELATED APPLICATIONS

This application is a continuation application of International Application Serial No. PCT/IB2015/053699 filed on May 20, 2015, which claims priority under 35 U.S.C. §119 of Indian Application No. 1694/MUM/2014 filed on May 20, 2014, the disclosures of which are hereby incorporated by reference in their entirety. The international application under PCT article 21(2) was published in English.

### FIELD OF THE DISCLOSURE

The present disclosure relates to a metal alkoxide, particularly magnesium alkoxide and a process for its preparation. This application also hereby incorporates the teachings of U.S. Pat. No. 8,633,124 to Gupta which issued on Jan. 21, 2014, the disclosure of which is hereby incorporated herein by reference in its entirety.

### BACKGROUND

Conventionally, magnesium alkoxide is prepared using magnesium metal and alcohol in the presence of iodine as an initiator. For instance, WO 2005/044873 discloses a method for synthesizing spherical magnesium alkoxide particles by reacting magnesium with an alcohol mixture at a temperature below the boiling point of the mixture. However, it is observed that the reaction time is 50 to 55 hrs, which renders the process inefficient.

Further, U.S. Pat. No. 4,959,336 discloses preparation of magnesium ethoxide in which magnesium powder is slurried with toluene and ethanol followed by addition of bromine and an ethanol solution of ferric chloride. It is found that after 20 minutes, the reaction mixture starts boiling. The obtained mixture is then kept for two days followed by addition of titanium isopropoxide and toluene and boiling at 105° C.

The process disclosed in said US patent involves tedious operative conditions. Further, the obtained magnesium alkoxide remains contaminated with bromine and iron chloride. Moreover, the obtained magnesium alkoxide is frangible and does not retain its morphology or particle size.

Therefore, there is felt a need to provide a process which will produce magnesium alkoxide having improved morphology including enhanced porosity and pore size distribution.

### OBJECTS

Some of the objects of the present disclosure, which at least one embodiment is able to achieve, are discussed herein below.

It is an object of the present disclosure to provide a magnesium alkoxide having micro pores in addition to macro and meso pores.

It is another object of the present disclosure to provide a novel process for preparing a magnesium alkoxide having micro pores in addition to macro and meso pores.

It is yet another object of the present invention to provide a magnesium alkoxide without initiator impurity.

Other objects and advantages of the present disclosure will be more apparent from the following description which is not intended to limit the scope of the present disclosure.

### SUMMARY

In accordance with one aspect of the present disclosure there is provided magnesium alkoxide characterized by:
a. mean particle size in the range of 20 to 70μ;
b. surface area in the range of 1 to 30 m²/g;
c. circularity in the range of 0.5 to 0.9;
d. macro pore size distribution in the range of 40 to 80%;
e. meso pore size distribution in the range of 15 to 60%; and
f. micro pore size distribution in the range of 2 to 10%.

In accordance with another aspect of the present disclosure there is provided a process for preparing a morphologically modified magnesium alkoxide, said process comprising the following steps:
a. contacting at a temperature in the range of 40 to 65° C. magnesium metal with at least one alcohol and at least one initiator for a time period of 30 min. to 3 hrs. to obtain a first mass;
b. heating and maintaining the first mass at a temperature in the range of 65 to 80° C. for a period of 30 min. to 10 hrs to obtain a second mass; and
c. drying the second mass under inert atmosphere at temperature in the range of 60 to 120° C. to obtain a morphologically modified magnesium alkoxide.

The alcohol can be at least one selected from the group consisting of methanol, ethanol, propanol, isopropanol, butanol, iso-butanol and a mixture of alcohols.

Typically, the molar ratio of magnesium metal to alcohol ranges from 1:2 to 1:20.

The initiator can be at least one selected form the group consisting of titanium tetrachloride and magnesium dichloride.

The step (a) can be carried out at a pressure ranging from 0.1 to 5 atm.

### DEFINITIONS

Macro porosity—Refers to pores greater than 50 nm in diameter.
Meso porosity—Refers to pores greater than 2 nm and less than 50 nm in diameter.
Micro porosity—Refers to pores smaller than 2 nm in diameter.
Pore size distribution and surface area is measured by nitrogen adsorption-desorption method by Sorptomatic 1990 instrument.
Circularity is measured by image-j software.
Brunauer-Emmett-Teller (BET) method is used for Surface Area Analysis and Barrett-Joyner-Halenda (BJH) method is used for Pore Size distribution.

### DETAILED DESCRIPTION

Magnesium alkoxide particles synthesized by conventional methods are frangible and do not retain their morphology or particle size during their application, especially when a pro-catalyst synthesis is carried.

The Indian Patent application No. 916/MUM/2008 (IN251446) discloses synthesis of spheroidal magnesium alkoxide having improved mechanical strength and narrow particle size distribution. The process involves reacting magnesium metal in the presence of iodine with a mixture of at least two alcohols. The process disclosed in the Indian Patent application No. 916/MUM/2008 utilizes iodine as an initiator which results in the formation of spheroidal magnesium alkoxide, which are devoid of micro pores. The inventors of the present disclosure particularly focused on modifying and improving the known process. Experiments were carried out to find substitutes for the known initiator, iodine. This substitute initiator can be effectively utilized to prepare magnesium alkoxide having micro pores in addition to macro and meso pores and the desired pore size distribution.

In the present disclosure, it is found that the initiators such as titanium tetra chloride and magnesium dichloride are suitable and effective to prepare magnesium alkoxide having micro pores.

In accordance with the present disclosure there is provided a novel process for preparing a morphologically modified magnesium alkoxide. The process involves the following steps:

In the first step, magnesium metal is contacted at a temperature in the range of 40 to 65° C. with at least one alcohol and at least one initiator for a period of 30 min. to 3 hrs. to obtain a first mass. Non limiting examples of alcohol include methanol, ethanol, propanol, isopropanol, butanol, iso-butanol and a mixture of alcohols. In accordance with the present disclosure, the ratio of magnesium metal to alcohol ranges from 1:2 to 1:20. The first step can be carried out at a pressure ranging from 0.1 to 5 atm.

The initiator used in the present disclosure includes but is not limited to titanium tetrachloride and magnesium dichloride.

In the second step, the first mass is heated and maintained at a temperature of 65 to 80° C. for a period of 30 min. to 10 hrs to obtain a second mass. Finally, the second mass is dried under inert atmosphere (nitrogen) at 60 to 120° C. to obtain the morphologically modified magnesium alkoxide.

The magnesium alkoxide prepared in accordance with the process of the present disclosure is characterized by the following characteristics:
i. mean particle size in the range of 20 to 70μ;
ii. surface area in the range of 1 to 30 m²/g;
iii. circularity in the range of 0.5 to 0.9;
iv. macro pore size distribution in the range of 40 to 80%;
v. meso pore size distribution in the range of 15 to 60%; and
vi. micro pore size distribution in the range of 2 to 10%.

The present disclosure is further described in the light of the following examples which are set forth for illustration purpose only and not to be construed for limiting the scope of the disclosure.

### EXAMPLE 1

### Preparation of the Morphologically Modified Magnesium Alkoxide using Titanium Tetrachloride

0.41 mole of magnesium was reacted with 4 mole of ethanol in the presence of 0.0027 mole of TiCl₄. The temperature of the obtained mass was increased from 20° C. to 80° C. for completion of the reaction. The magnesium alkoxide so produced was then dried at 100° C. under nitrogen.

### EXAMPLE 2

### Preparation of the Morphologically Modified Magnesium Alkoxide using Magnesium Dichloride

0.41 mole of magnesium was reacted with 4 mole of ethanol in the presence of 0.0212 mole of magnesium dichloride. The temperature of the obtained mass was increased from 20° C. to 80° C. for completion of the reaction. The magnesium alkoxide so produced was then dried at 100° C. under nitrogen.

TABLE 1
Comparative analysis for the magnesium alkoxide produced
using Iodine (I₂), titanium tetrachloride (TiCl₄) and
magnesium dichloride (MgCl₂) as initiators.

| | | Surface | Pore Size Distribution, | | | |
|---|---|---|---|---|---|---|
| | | Area | % | | | Circularity, |
| No. | Initiator | m²/g | Macro | Meso | Micro | Fraction |
| Conventional | I₂ | 32-38 | 50-60 | 40-50 | 0 | 0.68 ± 0.03 |
| Example 1 | TiCl₄ | 18-24 | 55-65 | 25-35 | 5-10 | 0.75 ± 0.03 |
| Example 2 | MgCl₂ | 22-28 | 60-70 | 20-30 | 2-8 | 0.68 ± 0.03 |

From Table 1, it is observed that the morphologically modified Mg-alkoxide prepared by using TiCl₄ or MgCl₂ initiator has improved morphology, including enhanced porosity and pore size distribution, as compared to the conventional Mg-alkoxide prepared by using I₂ initiator. The modified Mg-alkoxide of the present disclosure shows micro pores along with meso pores and macro pores. The highly porous morphologically modified Mg-alkoxide containing catalyst composition may be used in polymerization reactions to provide more porosity to the polymer resin.

### TECHNICAL ADVANCEMENT AND ECONOMIC SIGNIFICANCE

The present disclosure has the following advantages:
The morphologically modified magnesium alkoxide has improved morphology, including enhanced porosity and pore size distribution
The modified magnesium alkoxide shows micro pores along with meso pores and macro pores.
Catalyst compositions containing the highly porous morphologically modified magnesium alkoxide may be used in polymerization reactions to produce high rubber impact co-polymers, where high porosity homopolymer matrix is desired.

The embodiments herein and the various features and advantageous details thereof are explained with reference to the non-limiting embodiments in the description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the invention to achieve one or more of the desired objects or results.

The numerical values given for various physical parameters, dimensions and quantities are only approximate values and it is envisaged that the values higher than the numerical value assigned to the physical parameters, dimensions and quantities fall within the scope of the invention and the claims unless there is a statement in the specification to the contrary.

While certain embodiments of the inventions have been described, these embodiments have been presented by way of example only, and are not intended to limit the scope of the inventions. Variations or modifications in the process or compound or formulation or combination of this invention, within the scope of the invention, may occur to those skilled in the art upon reviewing the disclosure herein. Such variations or modifications are well within the spirit of this invention. The accompanying claims and their equivalents are intended to cover such forms or modifications as would fall within the scope and spirit of the invention.

1. Magnesium alkoxide comprising:
a. mean particle size in the range of 20 to 70μ;
b. surface area in the range of 1 to 30 m²/g;
c. circularity in the range of 0.5 to 0.9;
d. macro pore size distribution in the range of 40 to 80%;
e. meso pore size distribution in the range of 15 to 60%; and
f. micro pore size distribution in the range of 2 to 10%.

2. A process for preparing a morphologically modified magnesium alkoxide as claimed in claim 1, said process comprising the following steps:
a. contacting at a temperature in the range of 40 to 65° C. magnesium metal with at least one alcohol and at least one initiator for a time period of 30 min. to 3 hrs. to obtain a first mass;
b. heating and maintaining the first mass at a temperature in the range of 65 to 80° C. for a period of 30 min. to 10 hrs to obtain a second mass; and
c. drying the second mass under inert atmosphere at a temperature in the range of 60 to 120° C. to obtain a morphologically modified magnesium alkoxide.

3. The process as claimed in claim 2, wherein the alcohol is at least one selected from the group consisting of methanol, ethanol, propanol, isopropanol, butanol, iso-butanol and a mixture of alcohols.

4. The process as claimed in claim 2, wherein the molar ratio of magnesium metal to alcohol ranges from 1:2 to 1:20.

5. The process as claimed in claim 2, wherein said initiator is at least one selected form the group consisting of titanium tetrachloride and magnesium dichloride.

6. The process as claimed in claim 2, wherein the magnesium alkoxide is characterized by: mean particle size in the range of 20 to 70μ; surface area in the range of 1 to 30 m²/g; circularity in the range of 0.5 to 0.9; macro pore size distribution in the range of 40 to 80%; meso pore size distribution in the range of 15 to 60%; and micro pore size distribution in the range of 2 to 10%.

7. The process as claimed in claim 2, wherein the step (a) is carried out at a pressure ranging from 0.1 to 5 atm.
